(19) United States
(12) Patent Application Publication
(10) Pub. No.: US 2002/0111320 A1
(43) Pub. Date: Aug. 15, 2002

(54) Method for purification of acarbose

(76) Inventors: Vilmos Keri, Debrecen (HU); Lajos Deak, Debrecen (HU)

Correspondence Address: KENYON & KENYON, ONE BROADWAY, NEW YORK, NY, 10004, US

(21) Appl. No.: 09/924,271

(22) Filed: Aug. 7, 2001

Related U.S. Application Data
(60) Provisional application No. 60/223,492, filed on Aug. 7, 2000.

(51) Int. Cl. A61K 31/7036; C07H 5/04

(52) U.S. Cl. 514/42; 536/18.7

(57) ABSTRACT

The present invention relates to a novel process for the preparation of acarbose. Said process comprises the steps of: 1) acidifying a fermentation broth containing an acarbose; 2) removing particulates from the fermentation broth; 3) adsorbing the acarbose on a cation-exchanger in the presence of an anion of a weak acid; 4) eluting the acarbose from the cation-exchanger with at least one of hydrochloric acid and the weak acid; 5) precipitating the acarbose with a solvent; and 6) separating the acarbose.

### CROSS REFERENCE TO RELATED APPLICATION

This application claims the benefits of the Provisional Application Serial No. 60/223,492 filed Aug. 7, 2000, the disclosure of which is incorporated by reference in its entirety herein.

### FIELD OF THE INVENTION

The present invention relates to a novel process for the purification of acarbose.

### BACKGROUND OF THE INVENTION

Acarbose, also known as O-4,6-Dideoxy-4[[[1S-(1α,4α, 5β,6α)]-4,5,6-trihydroxy-3-(hydroxmethyl)-2-cyclohexen-1-yl]amino]-α-D-glycopyranosyl-(1→4)-O-α-D-glucopyranosyl-(1→4)-D-glucose, or 4″,6″-dideoxyl-4″-[(1S)-(1,4,6/5)-4,5,6-trihydrox-3-hydroxymethyl-2-cyclohexenylamino]maltotriose, having the following formula (I).

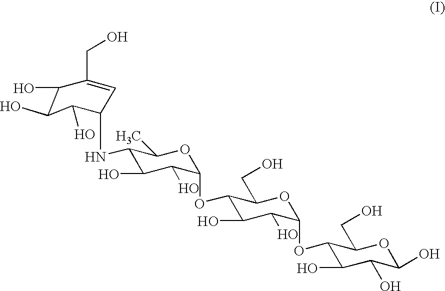

Acarbose is a potent α-glucosidase inhibitor that reduces sugar absorption in the gastrointestinal tract. It is used as an orally administered anti-diabetic drug sold under the trademark GLUCOBAY® and is available for the treatment of diabetes mellitus in humans.

U.S. Pat. No. 4,062,950 and Ger. Pat. No. 2,347,782 describe the isolation of acarbose from strains of Actinoplanes. These processes employ the use of ion-exchangers to adsorb acarbose from fermentation broths; but the ion-exchange steps contain nitrate anion. The presence of nitrate anion causes impurities to adsorb onto the ion-exchange resins and thus contaminates the acarbose. The presence of impurities also complicates the purification process because additional purification steps are needed to remove these impurities.

There is a need for an improved process for purification for acarbose. It is desirable to develop a purification process for acarbose whereby an increased purity of acarbose can be obtained with simplified purification steps.

### SUMMARY OF THE INVENTION

According to one aspect, the present invention provides a process for the purification of acarbose using ion-exchange chromatography; more specifically, cation-exchange chromatography.

According to another aspect, the present invention provides the use of a strong cation-exchange to adsorb acarbose in the presence of an anion of weak acid.

According to another aspect, the present invention provides a method of purifying acarbose, which comprises the steps of:

1) acidifying a fermentation broth containing an acarbose;

2) removing particulates from the fermentation broth;

3) adsorbing the acarbose on a cation-exchanger in the presence of an anion of a weak acid;

4) eluting the acarbose from the cation-exchanger with at least one of hydrochloric acid and the weak acid;

5) precipitating the acarbose with a solvent; and

6) separating the acarbose.

According to another aspect, the present invention provides a method of purifying acarbose, comprising the steps of:

1) acidifying a fermentation broth containing an acarbose;

2) removing particulates from the fermentation broth;

3) adsorbing the acarbose on an anion-exchanger in the presence of an anion of a weak acid;

4) eluting the acarbose from the anion-exchanger;

5) adsorbing the eluted acarbose on an cation-exchanger;

6) eluting the acarbose from the cation-exchanger in the presence of the anion of a weak acid;

7) precipitating the acarbose with a solvent; and

8) separating the acarbose.

### DESCRIPTION OF THE INVENTION

Definition

As used herein, the term “anion” refers to a negatively-charged ion and the term “cation” refers to a positively-charged ion.

As used herein, “ion exchange chromatography” refers to a charged ion-exchanger where it involves the binding and elution of a target molecule (e.g., acarbose).

As used herein, a “cation-exchanger” is a type of charged ion-exchanger that possesses a net negative charge on its resin which acarbose would binds to. One skilled in the art will appreciate that a strong ion-exchanger is one which remains almost fully ionized over a wide pH range whereas a weak exchanger is ionized over a small pH range. The terms “strong cation-exchanger” and “strong acid cation-exchanger” are used interchangeably and they refer to the same types of cation-exchangers.

Among the strong acid cationic exchange resins which may be used are those having sulfonic acid (SO3⁻H⁺) groups. These include the commercial products Amberlite® IR-118, IR-120, 252H; Amberlyst® 15, 36; Amberject® 1200 (H) (Rohm and Haas); Dowex® 50 wX series, Dowex® HCR-W2, Dowex® 650C, Dowex® Marathon C, Dowex® DR-2030, and Dowex® HCR-S, ion exchange resin (Dow Chemical Co.); Diaion® SK 102 to 116 resin series (Mitsubishi Chemical Corp.) And Lewatit SP 120 (Bayer). The preferred strong acid cationic exchange resins are Amberlite® 120, Dowex® 50 WX and Diaion® SK series.

Preferred cation-exchangers also include Amberlite®. Amerblite ion-exchanger employs a polystyrene resins as the matrix. Amberlite® 252 resin in H⁺ form is an example for cation-exchanger in H+ form. Preferred cation-exchanger is Amberlite® 252 in H⁺ form.

Cation ion-exchangers further include sulpho, sulphomethyl (i.e., methyl sulfonate), and sulphopropyl forms. Preferable cation-ion exchangers include the functional group of meththyl sulfonate. Exemplary strong cation-exchangers include Mini S® (methyl sulfonate), Mono S® (methyl sulfonate), SP Sepharose® (methyl sulfonate), SOURCE 15S®, 30S® (methyl sulfonate) and the like.

Weak cation ion-exchange resins include those which have carboxylic acid groups as well as carboxy and carboxymethyl forms. Preferable weak cation-exchangers include the functional group of —COOH. An exemplary weak cation-exchangers is CM Sepharose Fast Flow®.

As used herein, an “anion-exchanger” refers to anion-exchange resins that possess a net positive charge. A preferred anion-exchange resin include resins that contain a quarternary amine functional group. Diethylaminoethyl (DEAE) exchangers and carboxymethyl (CM) exchangers are usually used as anion exchangers.

As used herein, the term “an anion of a ak acid” refers to an anion of organic acids or phosphate. The anion of weak acid is selected from the group consisting of tartarate, succinate, citrate, acetate, formate, malonate, oxalate, phthalate, benzoate and phosphate.

As used herein, the term “weak acid” specifically refers to an acid selected from the group consisting of tartaric acid, succinic acid, citric acid, acetic acid, formic acid, malonic acid, oxalic acid, phthalic acid, benzoic acid and phosphoric acid.

As used herein, the term “particulates” refers to cellular debris and particles that are present in a fermentation broth. Particulates also include mycelium.

As used herein, the term “M” refers to a concentration in molar.

As used herein, the yield % is based on w/w. Each peak has an area on a HPLC chromatogram. “Area %” refers to the peak area of purified product divided by the total area of all peaks multiplied by 100.

As used herein, the term “yield of anion exchange” (See Table 1) refers to yield % of acarbose prior to cation-exchange column. Before the cation-exchange, anions were changed to an an anion of a weak acid (herein also known as “investigated anion”). This was achieved by a particular anion-exchanger.

As used here, the term “summarized yield” refers to anion-exchange yield multiplied by cation-exchange yield. Because anion exchangers generally have some non-specific absorption ability, it causes a loss.

According to one aspect, the present invention provides a process of purifying acarbose employing the use of a cation-exchanger. More specifically, the purification of acarbose using cation-exchanger in the presence of an anion of a weak acid.

According to another aspect, the present invention provides a process of purifying acarbose employing the presence of an anion of a weak acid during the cation-exchanger. When the anion of a weak acid is present, it is found that the impurities present in the fermentation broth cannot adsorb onto the strong acid cation-exchanger. Consequently, only acarbose adsorbs onto the strong acid cation-exchanger, and results in a better purification. This results in selective adsorption of acarbose. Accordingly, we found a novel phenomenon that adsorption of acarbose without the impurities.

According to another aspect, the present invention provides the acarbose adsorbing onto a strong acid cation-exchanger without previous desalting. In contrast, when counter-ions such as chloride, nitrate and the like are used, it is found that deslating is required.

According to another aspect, the present invention provides an unexpected phenomenon where it is found that the specific type of anion can influence the selectivity and adsorption capacity of the cation-exchanger.

According to one embodiment, the present invention provides a purification process for acarbose employing an appropriate anion which is selected from the group consisting of tartarate, succinate, citrate, acetate, formate, malonate, oxalate, phthalate, benzoate, and phosphate.

According to another embodiment, the present invention provides a process for purifying acarbose employing the use of multiple ion-exchangers. Fermentation broth is allowed to adsorb onto multiple ion-exchangers successively. In particular, acarbose is eluted from an anion-exchanger prior to the adsorption onto a cation-exchanger. The use of successive exchangers has proved to be effective in purifying acarbose.

A preferred embodiment for the anion-exchanger is an anion exchanger resin in OH⁻ form. A preferred embodiment for the anions used in the anion-exchange include tartarate, succinate, citrate, acetate, formate, malonate, oxalate, phthalate, benzoate, and phosphate.

A preferred embodiment for the cation-exchanger is a strong cation-exchanger. The presently most preferred embodiment includes a cation-exchanger that is a strong cation exchange resin in acid form.

According to another embodiment, the present invention employs a cation-exchanger whereby a strong cation-exchanger resin is in calcium form.

According to another embodiment, the particulates present in the fermentation broth are removed. The techniques to remove the particulates includes the sedimentation as well as filtering as one of skill in the art would appreciate. Fermentation broth containing acarbose can be filtered prior to the application onto the cation-exchangers. The filtration of fermentation broth removes any particulates and cell debris. Preferably, the filter is a pre-coat vacuum drum filter. One skilled in the art would appreciate the use of other filters of a similar kind and can serve a similar function as to pre-clear the fermentation broth prior to the chromatography purification. Most preferably, the filtration of fermentation broth is repeated at least twice.

According to another embodiment, the fermentation broth containing acarbose is adjusted to an acidic pH prior to filtration. Preferably, prior to the first filtration, the pH of the fermentation broth is adjusted to a pH of about 4.0 to a pH of about 6.0 with a mineral acid or a weak acid.

A mineral acid is defined herein as a strong acidic solution such as hydrochloric acid, sulphuric acid, nitric acid, phosphoric acid and the like.

A weak acid is selected from the group consisting of tartaric acid, succinic acid, citric acid, acetic acid, formic acid, malonic acid, oxalic acid, phthalic acid, benzoic acid, and phosphoric acid. A preferred embodiment for a weak acid is acetic acid.

According to another embodiment, the present invention relates to a process of purifying acarbose using two ion-exchangers. Preferably, the first ion-exchanger is an anion-exchanger. Most preferably, the first anion-exchanger is in the acetate, tartarate or succinate forms.

Preferably, the second ion-exchanger is a strong cation-exchanger. Most preferably, the second cation-exchanger is a strong cation-exchanger in acid form.

According to another embodiment, the present invention relates to a process of purifying acarbose, wherein acarbose adsorbed onto a cation-exchanger is eluted with either hydrochloric acid or weak acids.

According to another embodiment, the present invention relates to a process of purifying acarbose, wherein a solvent is used for the precipitation of acarbose from the eluant. Preferably the solvent includes alcohol, a mixture of alcohols and acetone, acetonitrile, ester of acetic acid, ester of formic acid, ester of propionic acid or the like.

The present invention is described in further detail with reference to the following examples. However, the present invention is by no means restricted to these specific examples.

### EXAMPLES

### Example 1

A fermentation broth of 122 kg was acidified with sulfuric acid to about pH 4.0-4.5. The acidified fermentation broth was filtered on pre-coat vacuum drum filter. The filtered mycelium was washed with water. The fermentation broth contained 537 gram active substance. The filtration yield was 91% (w/w). The volume of the filtrate was 227 liters.

The pH of the acidified filtrate was adjusted to about 2.0-2.2 with sulfuric acid and it was filtered again pre-coat drum filter. The volume of the filtrate was 223 liters. The filtration yield was 94% (w/w).

The pH of the filtrate of about 2.0-2.2 was adjusted to about 4.0-7.0 with anion-exchange resin in basic form. The yield of the pH adjust was 94.5% (w/w).

The adjusted filtrate was poured through on ion-exchange column. The ion-exchange column contained 20 liters anion-exchange resin in acetate form. The flow rate was 12.5 liters/hour. The effluent flow was conducted without desalinating continuously to another ion-exchange column containing 22 liters strong acid cation-exchanger in acid form. The ion-exchange was finished with 50 liters rinsing water.

The active substance that were bound or adsorbed onto the ion-exchange resin was eluted with 0.02 M hydrochloric acid. The eluants were collected into different fractions using a fraction collector. A main fraction of the eluants contained 374 gram active substance. The volume of the main fraction was 37.5 liters.

The summarized yield of the adsorption and elution was 87% (w/w).

The main fraction was analyzed by HPLC. HPLC method was as follows: Supercoil LC-NH₂ column; 5 μM; mobile phase: 1.2 gram KH₂PO₄ and 0.7 gram Na₂HPO₄ in 1,000 mL water; detection: UV2=210 nm. There was less than 10% related substances on HPLC. The pH of the main fraction was adjusted to about 4.0-5.0 with anion-exchange resin in basic form.

### Example 2

Another purification of acarbose was performed with the following procedures.

A part (480 mL) of the pH adjusted main fraction was taken for purification. This fraction contained 4.9 gram acarbose.

Two ion-exchange columns connected in series were used.

The first ion-exchange column contained 60 ml anion-exchange resin in tartarte form. The second column contained 60 ml strong acid cation-exchanger in acid form. The applied flow rate was 40 ml/hour. The ion-exchange was finished with 120 mL rinsing water.

The adsorbed active substance was eluted from the second column with 0.02 M hydrochloric acid. The main fraction contained 4.4 gram acarbose. The main fraction was analyzed by HPLC. There were less than 2% related substances on the HPLC chromatogram. The main fraction was concentrated after removing chloride ions with anion exchange resin in basic form. The concentration of acarbose was about 50% (w/w).

The acarbose was precipitated in the presence of ethanol. The crystals were filtered and dried. The 4 gram product contained less than 1% related substances.

### Example 3

Another purification of acarbose was performed with the following procedures.

A part (480 mL) of the pH adjusted main fraction (final solution of Example 1) was taken for purification. This part contained 4.8 gram acarbose.

Two ion-exchange columns connected in series were used.

The first ion-exchange column contained 60 mL anion-exchange resin in succinate form. The second column contained 60 mL strong acid cation-exchanger in acid form. The applied flow rate was 40 mL/hour. The ion-exchange was finished with 120 mL rinsing water.

The adsorbed active substance was eluted from the second column with 0.02 M hydrochloric acid. The main fraction contained 4.3 acarbose. The main fraction was analyzed with HPLC analysis method. There were less than 2% related substances on the HPLC chromatogram. The main fraction was concentrated after removing chloride ions with anion exchange resin in basic form. The concentration of acarbose was about 50% by w/w.

The acarbose was precipitated in the presence of ethanol. The crystals were filtered and dried. The 3.9 gram product contained less than 1% related substance.

### Example 4

The purification of acarbose illustrated in the above-mentioned Example 1 were using strong ion-exchanger in the presence of an anion of weak acids such as acetate, tartarte or succinate.

We found that other anion of weak acids can also influence the purification of acarbose during the ion-exchange chromatography. Table 1 summarizes the comparison of the efficiency of other anion of weak acids. Before the step of adsorbing acarbose onto the cation-exchanger, an anion exchanger was used to change the anion content of the filtrate from an existing anion (a stronger anion such as sulphate, chloride, nitrate and the like) to an anion of a weak acid.

Optimal effects of other anion of weak acids on the cation-exchange chromatography in acarbose purification is seen in Table 1.

### Example 5

A fermentation broth of 60 kg was acidified with acetic acid to Ph about 4.0-6.0. Acid was added to fermentation broth and mixed. The acidified fermentation broth was filtered on pre-coat vacuum drum filter. The filtered mycelium was washed with water. The fermentation broth contained 160 gram active substance. The filtration yield was 91% (w/w) using a HPLC method. The volume of the filtrate was 88 liters.

The filtrate was poured through on ion-exchange column. The ion-exchange column contained 8 liters strong acid cation-exchanger in acid form (Amberlite® 252 in H⁺ form). The ion-exchange was finished with 8 liters rinsing water.

The active substance that were bound or adsorbed onto the ion-exchange resin was eluted with 0.02 M hydrochloric acid. The flow-rate was 1 liter/hour. Preferred solution is hydrochloric acid. Preferred concentration is 0.0002 M-0.03 M. Most preferred concentration is 0.005 M-0.02 M. The eluants were collected into different fractions using a fraction collector. A main fraction of the eluants contained 124 gram active substance.

The yield of ion-exchange purification process was 85% w/w as determined by HPLC.

The main fraction was analyzed by HPLC. Acarbose had a purity of 94.5 area %. There were less than 10% impurity content. The details of HPLC were as follows: HPLC column used: Supercosil LC-NH₂; particle size: 5 μM; length: 250 mm; diameter: 4.6 mm; mobile phase: 1.2 gram KH₂PO₄ and 0.7 gram Na₂HPO₄ in 1,000 mL water (pH: 6.5); injection volume: 20 μL; and detection: UV2=210 nm.

It will be appreciated that the instant specification and claims are set forth by way of illustration and not limitation, and that various modifications and changes may be made without departing from the spirit and scope of the present invention.

TABLE 1
ACARBOSE
Effect of anions of a weak acid on cation-exchange
(Amberlite ® 252 resin in H⁺ form, 15 cm resin height, eluant: 0.02 N HCl in each case,
all fractions were combined)

| Anion of a weak acid | Borate | Tartarate | Succinate | Citrate | Acetate | Formate | Maleinate | Malonate | Oxalate |
|---|---|---|---|---|---|---|---|---|---|
| Sample name of solution | 292/376 | 292/377 | 292/378 | 292/378 | 292/380 | 292/381 | 292/382 | 292/383 | 292/384 |
| containing anion | | | | | | | | | |
| Acarbose (area %) in | 9.675 | 11.93 | 8.887 | 10.3 | 10.254 | 10.597 | 1.633 | 6.89 | 7.351 |
| the solution containing | | | | | | | | | |
| anions of weak acids | | | | | | | | | |
| Yield of anion | 97.0 | 94.9 | — | 95.4 | 96.2 | 100.0 | 99.5 | 100.0 | 100.0 |
| exchanger (%) | | | | | | | | | |
| Sample name of combined | 289/983 | 298/984 | 289/985 | 289/986 | 289/987 | 289/988 | 289/989 | 289/990 | 289/991 |
| fractions after cation- | | | | | | | | | |
| exchange | | | | | | | | | |
| Acarbose (area %) in | 31.15 | 76.95 | 74.486 | 68.395 | 71.280 | 70.102 | 7.639 | 55.437 | 68.533 |
| the combined fractions | | | | | | | | | |
| after cation-exchange | | | | | | | | | |
| Yield of cation- | 7.3 | 82.9 | — | 63.4 | 88.2 | 69.1 | 5.9 | 31.0 | 33.6 |
| exchanger (%) | | | | | | | | | |
| Summarized yield of the | 7.1 | 78.6 | 74.2 | 60.5 | 84.8 | 69.1 | 5.8 | 31.0 | 33.6 |
| two steps (%) | | | | | | | | | |

What is claimed is:

1. A process for purifying acarbose, comprising the steps of:
1) acidifying a fermentation broth containing an acarbose;
2) removing particulates from the fermentation broth;
3) adsorbing the acarbose on a cation-exchanger in the presence of an anion of a weak acid;
4) eluting the acarbose from the cation-exchanger with at least one of hydrochloric acid and the weak acid;
5) precipitating the acarbose with a solvent; and
6) separating the acarbose.

2. The process of claim 1, wherein the fermentation broth is acidified to a pH about 4 to about 6.

3. The process of claim 1, wherein the fermentation broth is acidified to a pH about 5.

4. The process of claim 1, wherein the fermentation broth is acidified with a weak acid.

5. The process of claim 4, wherein the weak acid is acetic acid.

6. The process of claim 4, wherein the weak acid is selected from the group consisting of tartaric acid, succinic acid, citric acid, formic acid, malonic acid, oxalic acid, phthalic acid, benzoic acid, phosphoric acid and the derivatives thereof.

7. The process of claim 1, wherein the particulates are removed with a filter.

8. The process of claim 7, wherein the filter is pre-coat vacuum drum.

9. The process of claim 1, wherein the cation-exchanger is a strong acid cation-exchanger.

10. The process of claim 9, wherein the strong acid cation exchanger is a resin in acid form.

11. The process of claim 1, wherein the anion of a weak acid is selected from the group consisting of tartarate, succinate, citrate, acetate, formate, malonate, oxalate, phthalate, benzoate, phosphate and the derivatives thereof.

12. The process of claim 1, wherein the acarbose is eluted from the cation-exchanger with hydrochloric acid.

13. The process of claim 12, wherein the hydrochloric acid has a concentration of about 0.002 M to about 0.03 M.

14. The process of claim 12, wherein the hydrochloric acid has a concentration of about 0.005 M to about 0.02 M.

15. The process of claim 1, wherein the acarbose is eluted from the cation-exchanger with a weak acid.

16. The process of claim 15, wherein the weak acid is selected from the group consisting of tartaric acid, succinic acid, citric acid, formic acid, malonic acid, oxalic acid, phthalic acid, benzoic acid, phosphoric acid and the derivatives thereof.

17. The process of claim 1, wherein the solvent used for precipitation is selected from the group consisting of alcohols, mixture of alcohols, acetone, acetonitrile, ester of acetic acid, ester of formic acid and ester of propionic acid.

18. A process for purifying acarbose, comprising the steps of:
1) acidifying a fermentation broth containing an acarbose;
2) removing particulates from the fermentation broth;
3) adsorbing the acarbose on an anion-exchanger in the presence of an anion of a weak acid;
4) eluting the acarbose from the anion-exchanger;
5) adsorbing the eluted acarbose on an cation-exchanger in the presence of the anion of a weak acid;
6) eluting the acarbose from the cation-exchanger;
7) precipitating the acarbose with a solvent; and
8) separating the acarbose.

19. The process of claim 18, wherein the fermentation broth is acidified to a pH about 2.0 to about 2.2.

20. The process of claim 18, wherein the fermentation broth is acidified with a mineral acid.

21. The process of claim 20, wherein the mineral acid comprises sulphuric acid, nitric acid, and phosphoric acid.

22. The process of claim 18, wherein the particulates are removed with a filter.

23. The process of claim 22, wherein the filter is pre-coat vacuum drum.

24. The process of claim 18, wherein the anion of a weak acid is selected from the group consisting of tartarate, succinate, citrate, acetate, formate, malonate, oxalate, phthalate, benzoate, phosphate and the derivatives thereof.

25. The process of claim 18, wherein the cation-exchanger is a strong acid cation-exchanger.

26. The process of claim 25, wherein the strong acid cation exchanger is a resin in acid form.

27. The process of claim 18, wherein acarbose is eluted from the cation-exchanger with a weak acid.

28. The process of claim 27, wherein the weak acid is selected from the group consisting of tartaric acid, succinic acid, citric acid, formic acid, malonic acid, oxalic acid, phthalic acid, benzoic acid, phosphoric acid and the derivatives thereof.

29. The process of claim 18, wherein the solvent used for precipitation is selected from the group consisting of alcohols, mixture of alcohols, acetone, acetonitrile, ester of acetic acid, ester of formic acid and ester of propionic acid.

30. The process of claim 20, wherein the fermentation broth is further acidified with an anion-exchanger.

31. The process of claim 30, wherein the anion-exchanger is resin in basic form.

32. The process of claim 30, wherein the fermentation broth is acidified to a pH about 4.0 to about 7.0.

33. The process of claim 18, wherein the solvent used for precipitation is selected from the group consisting of alcohols, mixture of alcohols, acetone, acetonitrile, ester of acetic acid, ester of formic acid and ester of propionic acid.

34. The process of claim 33, wherein the alcohol is selected from the group consisting of methanol, propanol and isopropanol and ethanol.
